(19) United States
(12) Patent Application Publication
(10) Pub. No.: US 2013/0090654 A1
(43) Pub. Date: Apr. 11, 2013

(54) COMBINATION CYSTOTOME AND ACCESS NEEDLE DEVICE AND METHOD

(71) Applicant: COOK MEDICAL TECHNOLOGIES LLC, Bloomington, IN (US)

(72) Inventor: Michael S. CLANCY, Limerick (IE)

(73) Assignee: COOK MEDICAL TECHNOLOGIES LLC, Bloomington, IN (US)

(21) Appl. No.: 13/633,418

(22) Filed: Oct. 2, 2012

Related U.S. Application Data
(60) Provisional application No. 61/545,343, filed on Oct. 10, 2011.

(51) Int. Cl. A61B 18/18

(52) U.S. Cl. 606/45

(57) ABSTRACT

A system and method are provided for accessing and cannulating a target mass. The system includes a combined cystotome and access needle that provides for efficient operation. The access needle may be embodied as a polymeric sheath with a piercing stylet configured to extend beyond its distal end. The device embodiments allow a user to access and cannulate a target site such as, for example, a pancreatic pseudocyst with a single device. The device is also configured to allow introduction of a wire guide such that the cannulated target site can readily receive a wire-guided stent, or a stent delivered over the access needle.

### CROSS-REFERENCE TO RELATED APPLICATIONS

This application claims priority to U.S. Provisional Application Ser. No. 61/545,343, filed Oct. 10, 2011, which is incorporated herein by reference in its entirety.

### TECHNICAL FIELD

The invention relates generally to methods of minimally-invasive surgical methods. More particularly, the invention pertains to a system and method for access to and cannulation of a body structure.

### BACKGROUND

The development of minimally invasive methods and devices over recent years has revolutionized the practice of medicine. These methods and devices allow clinicians to perform a wide variety of procedures while minimizing trauma to the patient. Along these lines, there is a need for devices and methods that employ minimally invasive technologies in order to access occluded regions in a mammalian body.

Cystotomy procedures are used in a variety of surgical procedures. For example, various ailments associated with the biliary tree can be treated by placement of a stent. One example is pancreatic pseudocysts, which may form in the pancreas as a pocket holding necrotic tissue, blood, and/or pancreatic secretions. They may occur, for example, in connection with pancreatitis or as the result of abdominal injury, and are differentiated from true cysts by being contained by a fibrous and/or granular tissue capsule rather than an epithelial lining. Pancreatic pseudocysts may be treated by accessing, cannulating, and placing a drainage stent such as, for example, a pigtail polymer stent into the pseudocyst. Cystotomy may also be used, for example, during treatment and/or removal of accretions (e.g., “stones”) from the urinary bladder.

Commonly, a pancreatic pseudocyst, or other structure in need of access for diagnostic and/or therapeutic purposes presently requires multiple steps and tools. Typically, one or more cannulation and/or dilator devices must be introduced to open the stricture, penetrate the pseudocyst, or otherwise provide a sufficient path of access for stent-introduction. The present procedure for placement of a drainage catheter into a pancreatic pseudocyst by cystotomy includes several steps that may be implemented during ERCP (Endoscopic retrograde cholangio-pancreatography).

First a metal access needle often including a stylet through its needle lumen is provided and directed to (and potentially into) the target area. Next the stylet is removed from the metal access needle, and a wire guide is directed through the needle lumen to and/or into the target site. Then, the access needle is removed, being pulled out over the entire length of the wire guide. Next, a separate cystotome is provided, and is directed along the entire length of the wire guide to the target site. A tissue cautery/electrosurgical element disposed at the distal tip of the cystotome is actuated by directing current therethrough to cannulate the target site with sufficient diameter for placement of a stent. Then, the cystotome is removed by being withdrawn along the entire wire guide length, and the drainage stent is directed over the wire guide into the target site. This is a painstaking and time-consuming procedure.

Therefore, it would be beneficial to provide minimally invasive access devices and methods that will decrease the number of steps taken, the number of items that treating personnel (e.g., physician and assisting personnel) must handle during a procedure, and the amount of time needed to complete a procedure such as, for example, endoscopically placing a drainage stent into a pancreatic pseudocyst.

### BRIEF SUMMARY

A device and method are provided including system and method for cannulating a target structure in a patient body.

### BRIEF DESCRIPTION OF THE DRAWINGS

The invention may be better understood with reference to the following drawings and description. The components in the figures are not necessarily drawn to scale, emphasis instead being placed upon illustrating the principles of the invention. In particular, by way of illustrative example, the components of the embodiments described here preferably will have a closer fit/tolerance than is shown in the drawings.

FIG. 1 shows an embodiment (in longitudinal section, showing a distal end length) of a system for accessing and cannulating a body structure; and

FIG. 2 shows another embodiment (in longitudinal section, showing a distal end length) of a system for accessing and cannulating a body structure.

### DETAILED DESCRIPTION

As used herein, including in the claims, the term “echogenic” is defined as having enhanced echogenicity. Specifically, it is used to refer to materials or portions of materials that are constructed or are treated to have greater reflectivity of ultrasonic waves than standard materials used for a stent, sheath, cannula, catheter, and/or stylet, and to provide an echogenic profile relative to surrounding tissues during use in a patient body to accurately orient and direct the echogenic device portion. It is known in the art that most materials used for a stent sheath, catheter, cannula, or stylet will reflect some ultrasonic waves, but the term “echogenicity,” as used herein includes treating the surface by creating a textured or patterned surface including, for example, one or more of dimples, divots, knurling, ridges, or the like—each of which is known in the art to enhance echogenicity as compared to a smooth surface for a similarly-sized/shaped object, (and/or, when specifically referenced, using a material known to provide an enhanced echogenic profile) configured to provide clear ultrasound visualization at a resolution providing for accurate location and navigation of a device in a body (e.g., of a patient). Echogenic construction may be enhanced by surface texture, but can also be provided by structural inclusions such as embedded bubbles, beads, or other inclusions in a polymer or metal that can provide for a different ultrasound reflectivity than material surrounding them.

Also, as used herein, the term “needle” refers generally to a tubular cannula that may or may not have a piercing distal tip, and the term “cannula” may refer to a rigid or flexible tubular device that may include a piercing tip. As used throughout unless a special exception is specifically identified, the term “proximal” refers to an end or direction nearer a physician or other person handling an object during normal use (generally, the “handle end”), and “distal” refers to the opposite end (generally, the “tool end”). For purposes of the present application, the term “cystotome” is defined by the structures and limitations of the present description and claims. Generally, a “cystotome” refers to a device for cystotomy of the urinary bladder, but those of skill in the art will appreciate that the device embodiments of the present disclosure will be useful in other procedures for making an incision and/or access aperture through a body structure of (including within) a patient's body. Drawing figures are not necessarily to scale, as various parts thereof may be magnified or otherwise emphasized to clarify structural features (including that, as one specific example, the proportions of the duodenum and biliary tree structure are not shown to scale, as those of skill in the art will know that the biliary tree is proportionally smaller than shown).

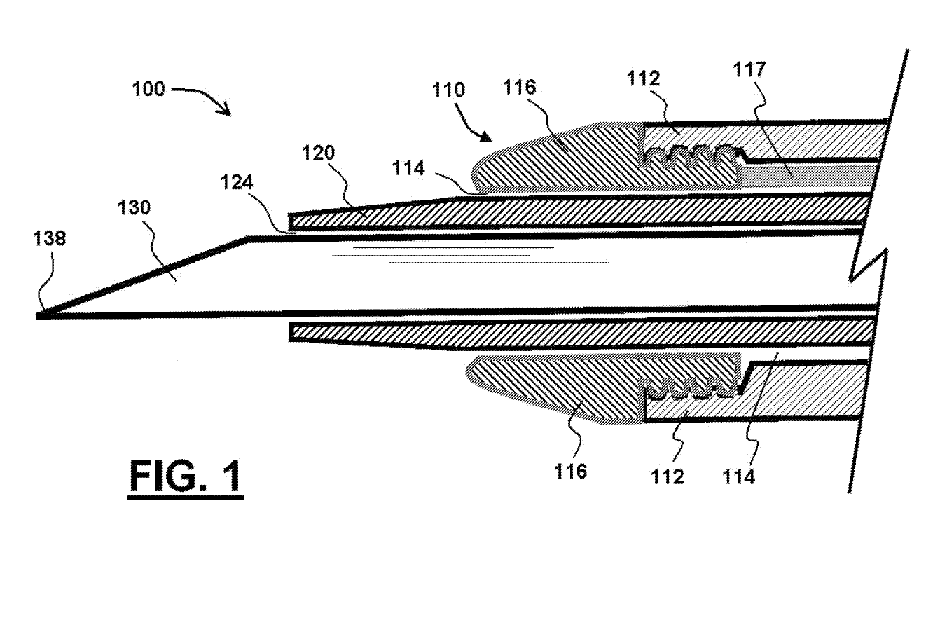

One embodiment of a system for accessing a body structure is described here with reference to FIG. 1, which shows a distal end portion of system components in longitudinal section. The system embodiment provides a combined cystotome and access needle device 100 that provides for a more efficient method of cannulation and access to a body structure such as a pancreatic pseudocyst. Although it is not explicitly illustrated in the drawings, a preferred device body will be configured with sufficient length, flexibility, pushability, and trackability to be operable through a working channel of a gastrointestinal endoscope (e.g., end-viewing and/or side-viewing scopes, including endoscopic ultrasound (EUS) endoscopes). Those of skill in the art will appreciate that several proximal end constructions of a handle and/or other operative proximal end structure may use or readily be adapted from existing devices, and practiced within the scope of the present invention. One or more of the cystotome, access needle sheath, and stylet components in any of the embodiments described below may be configured to include echogenic enhancement(s) for EUS visualization.

The device 100 includes a cystotome 110 that includes a flexible elongate tubular cystotome sheath body 112 having a distal end. The cystotome body 112 circumferentially defines a cystotome lumen 114 that extends longitudinally through its length. The cystotome 110 also includes a diathermic element 116 configured as a conductive ring that circumferentially defines the distal end of the cystotome 110 at the distal end of the cystotome body 112. The diathermic element 116 is configured in electrical communication via an electroconductive element embodied here as a wire 117 connected with a proximal region (not shown) of the cystotome that preferably will be configured to transmit energy (e.g., RF energy, electric current) from an electrosurgical power supply. The wire 117 may be electroinsulated.

The diathermic ring 116 is tapered with a rounded distal transition to the cystotome lumen 114. More particularly, the shape of the ring 116 may be described as including a tapered external geometry comprising at least one frustoconical length between a proximal diathermic element end and a distal diathermic element end. In some embodiments, the diathermic element 116 may be sized at about 6 Fr to about 10 Fr (about 2 to about 3.33 mm outer diameter), or larger or smaller for various applications. The diathermic element 116 is shown as connecting to the cystotome body by a threaded connection, but it should be appreciated that other connecting means (e.g., one or more of friction-fit, adhesive, sonic welding, or other connecting means) may be used to securely fix the diathermic element 116 to the cystotome body 112. The cystotome body 112 most preferably is constructed essence of a non-conductive material that will generally prevent conduction from the wire 117 through the body 112, thereby limiting the exposed conductive surface of the distal portion of the device 110 to the diathermic element.

An elongate, flexible access sheath, which may be configured as a tubular polymer access needle 120, is longitudinally and slidably disposed through the cystotome lumen 114. One suitable polymer for construction of at least a length of the access needle sheath 120 is polytetrafluoroethylene (PTFE). The access sheath 120 includes a needle lumen 124. The sheath 120 is configured with sufficient length to be advanced beyond the distal end of the cystotome 110 to provide for initial penetration of a target. A stylet 130, which may be configured as a polymer or a metal alloy stylet, is longitudinally, slidably, and removably through the needle lumen 124. The stylet 130 preferably includes a distal piercing tip 138.

During a method of using the device 100, an endoscope may be provided and directed to a predetermined location near a target site, such as—for example—a target mass of tissue, to be accessed and cannulated using the device 100. The stylet piercing tip 138 may be aligned adjacent and distal of the distal end of the access sheath 120. The device 100 may be directed through a working channel of the endoscope to the target site, and the needle 120 with stylet 130 may be advanced distally (extending past the diathermic element 116) to penetrate the target site. The cystotome 110 may then be advanced distally along the needle 120 until the diathermic element 116 contacts the target site around the penetrated locus. Then, the diathermic element 116 may be actuated (e.g., by directing electrosurgical current therethrough) to cannulate the target site).

The stylet 130 may be removed from the needle sheath lumen 124 and replaced with a wire guide (not shown). Or the needle sheath 120 may be removed from the cystotome lumen 114 and replaced with a wire guide (not shown). The cystotome 110 may be withdrawn proximally along and removed from the needle sheath 120 and/or the wire guide thereafter. Then a drainage stent may be directed along needle sheath 120 and/or the wire guide into the cannulated target site.

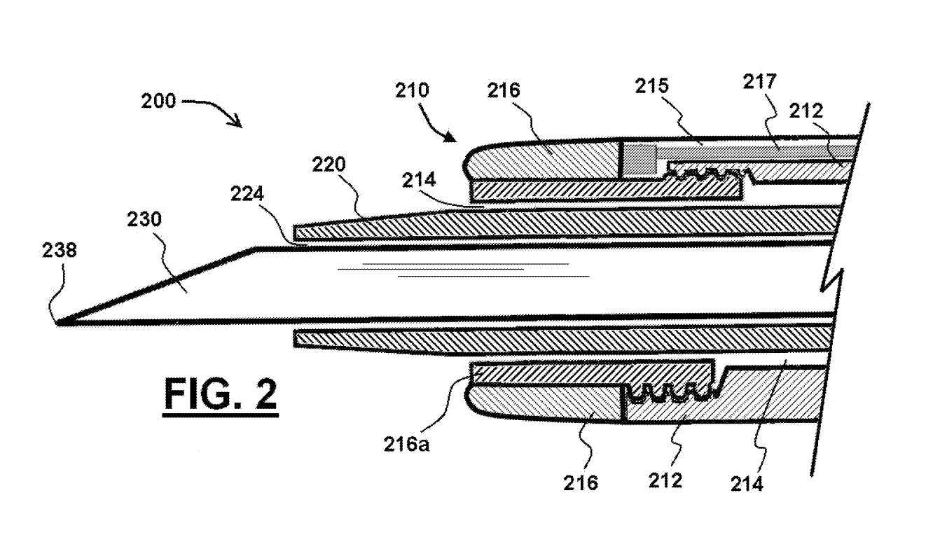

Another embodiment of a system for accessing a body structure is described here with reference to FIG. 2, which shows a distal end portion of system components in longitudinal section. The system embodiment provides a combined dual-lumen cystotome and access needle device 200 that provides for a more efficient method of cannulation and access to a body structure such as a pancreatic pseudocyst (e.g., during an endoscopic transgastric pancreatic necrosectomy procedure).

Although it is not illustrated, a preferred device body will be configured with sufficient length, flexibility, pushability, and trackability to be operable through a working channel of a gastrointestinal endoscope (end-viewing and/or side-viewing, including endoscopic ultrasound (EUS) endoscopes). Those of skill in the art will appreciate that several proximal end constructions of a handle and/or other operative proximal end structure may use or readily be adapted from existing devices, and practiced within the scope of the present invention.

The device 200 includes a cystotome 210 that includes a flexible elongate tubular cystotome sheath body 212 having a distal end. The cystotome body 212 circumferentially defines a first cystotome lumen 214 that extends longitudinally through its length, and a second cystotome lumen 215 that also extends longitudinally through its length. The cystotome 210 also includes a diathermic element 216 configured as a ring that circumferentially defines the distal end of the cystotome 210 at the distal end of the cystotome body 212. The diathermic element 216 is configured in electrical communication via an electroconductive element embodied here as a wire 217 that extends through the second cystotome lumen 215 and is connected with a proximal region (not shown) of the cystotome that preferably will be configured to transmit energy (e.g., RF energy, electric current) from an electrosurgical power supply.

The diathermic ring 216 is domed with a rounded distal transition from the outer surface of the cystotome sheath 210 to the cystotome lumen 214. More particularly, the shape of the ring 216 may be described as including a domed external geometry comprising at least two radii of curvature between a proximal diathermic element end and a distal diathermic element end. In some embodiments, the diathermic element 216 may be sized at about 6 Fr to about 10 Fr (about 2 to about 3.33 mm outer diameter), or larger or smaller for various applications. The diathermic element 216 is separated from the cystotome lumen by a tubular insulating ring 216a. The insulating ring 216a is shown as connecting to the cystotome body by a threaded connection, but it should be appreciated that other connecting means (e.g., one or more of friction-fit, adhesive, sonic welding, or other connecting means) may be used to securely fix the diathermic element 216 and the insulating ring 216a to the cystotome body 212. The cystotome body 212 most preferably is constructed essentially of a non-conductive material that will generally prevent conduction from the wire 217 through the body 212, thereby limiting the exposed conductive surface of the distal portion of the device 210 to the diathermic element, and preventing undesired transmission of energy from the wire 217 to the outside of the device and to the contents of the cystotome lumen 214.

An elongate, flexible access sheath, which may be configured as a tubular polymer or metallic access needle 220, is longitudinally and slidably disposed through the cystotome lumen 214. The access sheath 220 includes a needle lumen 224. The sheath 220 is configured with sufficient length to be advanced beyond the distal end of the cystotome 210 to provide for initial penetration of a target. A stylet 230, which may be configured as a polymer or a metal stylet, is longitudinally, slidably, and removably through the needle lumen 224. The stylet 230 preferably includes a distal piercing tip 238. Methods of use for this embodiment and variants thereof will be similar to the methods described above with reference to the embodiment of FIG. 1.

A further embodiment may provide a surgical procedure kit that may include an embodiment of FIG. 1, FIG. 2, or some variant thereof. Such a kit may further include one or more of at least one wire guide, at least one drainage stent, and/or at least one electrosurgical power supply connector configured to connect an electrosurgical power supply to the electroconductive structure providing electrical communication with the distal diathermic element of the cystotome.

Those of skill in the art will appreciate that embodiments not expressly illustrated herein may be practiced within the scope of the present invention, including that features described herein for different embodiments may be combined with each other and/or with currently-known or future-developed technologies while remaining within the scope of the claims presented here. It is therefore intended that the foregoing detailed description be regarded as illustrative rather than limiting. And, it should be understood that the following claims, including all equivalents, are intended to define the spirit and scope of this invention.

I claim:

1. A system for accessing and cannulating a body structure, the system comprising:
a cystotome including
an elongate flexible body having a distal end,
a cystotome lumen extending longitudinally through the cystotome body, and
a diathermic element disposed upon the distal end of the cystotome body, the diathermic element being configured in electrical communication with a proximal region of the cystotome body; and
an elongate flexible polymer access needle disposed longitudinally through the cystotome lumen, the polymer access needle comprising
a longitudinal needle lumen disposed through its length, and
an elongate flexible stylet disposed longitudinally, slidably, and removably through the needle lumen, the stylet including a distal piercing end.

2. The system of claim 1, wherein the electrical communication is provided by an electroconductive element disposed through a second longitudinal lumen of the cystotome body.

3. The system of claim 1, where the cystotome body is constructed essentially of non-electroconductive material.

4. The system of claim 1, where the cystotome body is constructed essentially of non-electroconductive material and includes a second longitudinal lumen, through which an electroconductive element is disposed and configured to provide the electrical communication.

5. The system of claim 1, where the stylet is constructed essentially of a metal alloy.

6. The system of claim 1, where the needle lumen is dimensioned for passage therethrough of a wire guide.

7. The system of claim 1, where the diathermic element is constructed as a ring defining a distal end of the cystotome lumen.

8. The system of claim 1, where the diathermic element is configured with a domed external geometry comprising at least two radii of curvature between a proximal diathermic element end and a distal diathermic element end.

9. The system of claim 1, where the diathermic element is configured with a tapered external geometry comprising at least one frustoconical length between a proximal diathermic element end and a distal diathermic element end.

10. The system of claim 1, wherein at least one length of the polymer access needle comprises polytetrafluoroethylene (PTFE).

11. The system of claim 1, wherein the system is dimensioned and configured with sufficient length and flexibility to operate through a working channel of a gastrointestinal endoscope.

12. A surgical procedure kit comprising:
the system of claim 1; and
a wire guide configured with sufficient length to operate through the needle lumen.

13. The kit of claim 12, further comprising at least one drainage stent.

14. The kit of claim 12, further comprising at least one electrosurgical power supply connector.

15. A system for accessing and cannulating a body structure, the system comprising:
a cystotome portion constructed to include an elongate flexible body having a distal end, a cystotome lumen extending longitudinally through the cystotome body, and a diathermic element disposed upon the distal end of the cystotome body, the diathermic element shaped as a ring defining a distal end of the cystotome lumen and configured in electrical communication with a proximal region of the cystotome body; and
an elongate flexible polymer access needle disposed longitudinally through the cystotome lumen, the polymer access needle including a needle lumen disposed longitudinally through its length, said needle lumen configured to receive a wire guide, and
an elongate flexible metal stylet disposed longitudinally, slidably, and removably through the needle lumen, the stylet including a distal piercing end.

16. A method for accessing a target mass in a patient body, the method comprising:
providing a system according to claim 15;
aligning the stylet distal piercing end adjacent and distal of a distal end of the polymer access needle;
directing the stylet distal piercing end and the distal end of the polymer access needle to a target mass;
withdrawing the stylet distal piercing end into the needle lumen;
directing the cystotome portion distally along the access needle to contact the target mass with the diathermic element; and
actuating the diathermic element to cannulate a portion of the target mass.

17. The method of claim 16, further comprising a step of removing the stylet.

18. The method of claim 17, further comprising a step of directing a wire guide through the needle lumen after the step of removing the stylet.

19. The method of claim 18, further comprising steps of:
after the step of actuating the diathermic element, removing the cystotome and the access needle from the wire guide; and
directing a drainage stent distally along the wire guide to the cannulated portion of the target mass

20. The method of claim 16, further comprising a step of removing the cystotome.
